(19) United States
(12) Patent Application Publication
(10) Pub. No.: US 2010/0056920 A1
(43) Pub. Date: Mar. 4, 2010

(54) ULTRASOUND SYSTEM AND METHOD OF PROVIDING ORIENTATION HELP VIEW

(75) Inventors: Sung Hee Kim, Seoul (KR); Yun Jin Kim, Seoul (KR)

Correspondence Address: JONES DAY, 222 EAST 41ST ST, NEW YORK, NY, 10017, US

(73) Assignee: Korea Institute of Science and Technology

(21) Appl. No.: 12/615,949

(22) Filed: Nov. 10, 2009

(30) Foreign Application Priority Data
Feb. 12, 2008 (KR) 10-2008-0121179
Jan. 30, 2009 (KR) 10-2009-0007198

(51) Int. Cl. A61B 8/14

(52) U.S. Cl. 600/443

(57) ABSTRACT

The present invention relates to an ultrasound system and a method of providing orientation help (OH). The ultrasound system comprises: an ultrasound data acquisition unit including an ultrasound probe for transmitting ultrasound signals to a target object and receiving ultrasound echo signals reflected from the target object, the ultrasound data acquisition unit being configured to acquire ultrasound data based on the ultrasound echo signals, the ultrasound data acquisition unit being further configured to detect an orientation and a scanning direction of the ultrasound probe; and a processor for forming a volume data based on the ultrasound data and forming an orientation help (OH) view showing a spatial orientation of volume data on a display region, the OH view including a probe orientation marker indicating the detected orientation of the ultrasound probe and a scanning direction marker indicating the detected scanning direction.

The present application claims priority from Korean Patent Application Nos. 10-2009-0007198 (filed on Jan. 30, 2009) and 10-2008-0121179 (filed on Dec. 2, 2008), the entire subject matters of which are incorporated herein by reference.

### BACKGROUND OF THE INVENTION

1. Technical Field

The present invention generally relates to ultrasound systems, and more particularly to an ultrasound system and a method of providing an orientation help (OH) view.

2. Background Art

The ultrasound system has become an important and popular diagnostic tool due to its non-invasive and non-destructive nature. Modern high-performance ultrasound imaging diagnostic systems and techniques are commonly used to produce two or three-dimensional images of internal features of patients (target objects).

Generally, the ultrasound system may provide a three-dimensional ultrasound image including clinical information such as spatial information and anatomical figures of the target objects, which cannot be provided by a two-dimensional ultrasound image.

The ultrasound system may provide an orientation help (OH) function to form an OH view showing a spatial orientation of volume data on a display region. The OH view may be three-dimensionally formed by using entire contours of the ultrasound volume data. The OH view may three-dimensionally indicate a position of reference plane, which may be selected by a user in the volume data, on a three-dimensional space.

In the conventional OH function, only the reference plane may be rotatable. That is, geometrical operations of the OH view such as rotation, reduction, enlargement, movement and the like may not be allowed. Thus, there is a problem since it is difficult for the user to recognize a spatial orientation of the volume data and the reference plane.

### BRIEF DESCRIPTION OF THE DRAWINGS

FIG. 1 is a bock diagram showing an illustrative embodiment of an ultrasound system.

FIG. 2 is a block diagram showing an ultrasound data acquisition unit.

FIG. 3 is a schematic diagram showing a volume data.

FIG. 4 is a block diagram showing an illustrative embodiment of a processor.

FIG. 5 is a schematic diagram showing an example of an orientation help view.

### DETAILED DESCRIPTION OF THE INVENTION

A detailed description may be provided with reference to the accompanying drawings. One of ordinary skill in the art may realize that the following description is illustrative only and is not in any way limiting. Other embodiments of the present invention may readily suggest themselves to such skilled persons having the benefit of this disclosure.

FIG. 1 is a block diagram showing an illustrative embodiment of an ultrasound system. The ultrasound system 100 may include an ultrasound data acquisition unit 110, a user interface 120, a processor 130 and a display unit 140.

The ultrasound data acquisition unit 110 may be configured to transmit ultrasound signals to a target object and receive ultrasound echo signals reflected from the target object. The ultrasound data acquisition unit 110 may be further configured to form ultrasound data of the target object based on the received ultrasound echo signals.

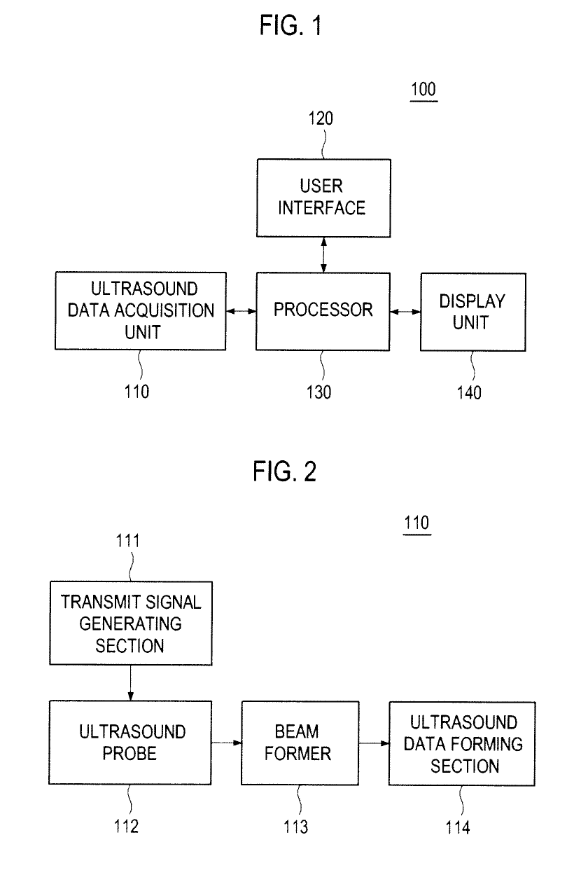

FIG. 2 is a block diagram showing the ultrasound data acquisition unit 110. The ultrasound data acquisition unit 110 may include a transmit (TX) signal generating section 111, an ultrasound probe 112 including a plurality of transducer elements (not shown), a beam former 113 and an ultrasound data forming section 114.

The TX signal generating section 111 may be operable to generate TX signals according to an image mode set in the ultrasound system 100. The image mode may include a brightness (B) mode, a Doppler (D) mode, a color flow mode, etc. In one embodiment, it will be described that the B mode is set in the ultrasound system 100 to obtain a B-mode image.

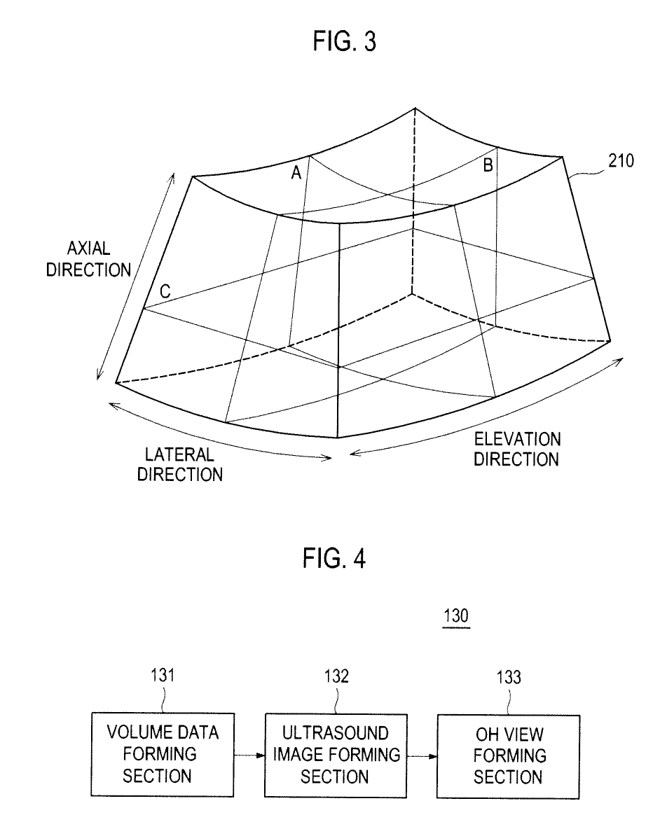

The ultrasound probe 112 may include a three-dimensional probe containing an array transducer having a plurality of transducer elements, which iteratively swing at a prescribed angle along an elevation direction as illustrated in FIG. 3. FIG. 3 is a schematic diagram showing a volume data. The details of FIG. 3 will be explained later.

The ultrasound probe 112 may be operable to transmit the ultrasound signals, which may travel into the target object, along scan lines set in an axial direction responsive to the TX signals received from the TX signal generating section 111. The ultrasound probe 112 may be further operable to receive ultrasound echo signals reflected from the target object and convert them into electrical receive signals. The ultrasound probe 112 may include an orientation detecting section (not shown) for detecting an orientation of the ultrasound probe 112 and a scanning direction of the ultrasound probe 112. In one embodiment, the scanning direction may indicate at least one of a scan line direction and a swing direction of the array transducer.

The beam former 113 may be operable to convert the electrical receive signals outputted from the ultrasound probe 112 into digital signals. The beam former 113 may be further operable to apply delays to the digital signals in consideration of the distances between the transducer elements and focal points to thereby output receive-focused signals.

The ultrasound data forming section 114 may be operable to form an ultrasound data of the target object based on the receive-focused signals. The ultrasound data forming section 114 may be further operable to perform signal processing upon the receive-focused signal such as gain adjustment, filtering and the like.

Referring back to FIG. 1, the user interface 120 may include at least one of a control panel (not shown), a mouse (not shown), a keyboard (not shown) and the like. The user interface 120 may be operable to allow a user to input user instructions. The user instructions may include first, second and third instructions. The first instruction may include setting a region of interest (ROI). The ROI setting may include size and position setting of the ROI. The second instruction may include selecting one of the reference planes. In one embodiment, by way of non-limiting examples, the reference planes may include an A plane, a B plane and a C plane in a volume data 210, as illustrated in FIG. 3. The third instruction may include setting a geometrical operation upon an orientation help (OH) view. The geometrical operation of the OH view may include one of rotation, reduction, enlargement and movement of the OH view.

Referring to FIG. 4, the processor 130 may include a volume data forming section 131, an ultrasound image forming section 132 and an OH view forming section 133. The volume data forming section 131 may be configured to form the volume data based on the ultrasound data. FIG. 3 is a schematic diagram showing a volume data. In FIG. 3, the elevation direction may be the swing direction of the array transducer, the axial direction may be the scan line direction of the array transducer and the lateral direction may be the longitudinal direction of the array transducer.

The ultrasound image forming section 132 may be configured to form the ultrasound images based on the volume data. The ultrasound image forming section 132 may form a three-dimensional ultrasound images as well as images of the reference planes corresponding to the A plane, the B plane and the C plane using the volume data 210 as illustrated in FIG. 3. The OH view forming section 133 may be configured to form an OH view 300 in response to the first and second user instructions.

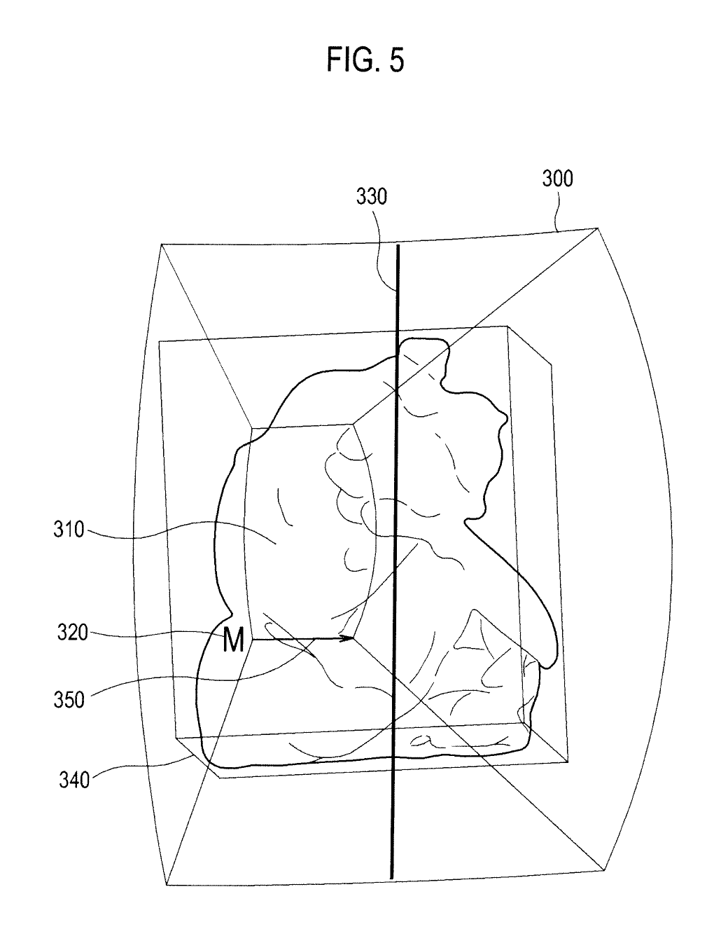

FIG. 5 is a schematic diagram showing an example of an orientation help view. Referring to FIG. 5, the OH view 300, which may show a spatial orientation of the volume data, includes the three-dimensional ultrasound image 310, a probe orientation marker 320 indicating a position of the ultrasound probe 112, a clip plane 330 indicating the images of the selected reference plane, a ROI 340 and a scanning direction marker 350.

In one embodiment, the probe orientation marker 320 may be formed based on the orientation of the ultrasound probe 112 outputted from the orientation detecting section of the ultrasound probe 112. Further, the scanning direction marker 350 may be formed based on the scanning direction outputted from orientation detection section of the ultrasound probe 112. The three-dimensional ultrasound image 310 may be overlapped with the OH view 300. In one embodiment, by way of non-limiting examples, the scanning direction marker 350 may include a first scanning direction marker indicating a swing direction (the elevation direction as illustrated in FIG. 3) of the array transducer and a second scanning direction marker indicating a scan line direction (the axial direction as illustrated in FIG. 3). The OH view forming section 133 may set the geometrical operation such as rotation, reduction, enlargement and movement of the OH view 300 in response to the third user instruction.

Referring back to FIG. 1, the display unit 140 may display the OH view 300, which is overlapped with the selected image of the reference planes and display the images of the A, B and C planes. The display unit 140 may further display on the screen the three-dimensional ultrasound image together with the images of the reference planes.

Any reference in this specification to “one embodiment,” “an embodiment,” “example embodiment,” “illustrative embodiment,” etc. means that a particular feature, structure or characteristic described in connection with the embodiment is included in at least one embodiment of the present invention. The appearances of such phrases in various places in the specification are not necessarily all referring to the same embodiment. Further, when a particular feature, structure or characteristic is described in connection with any embodiment, it is submitted that it is within the purview of one skilled in the art to affect such feature, structure or characteristic in connection with other ones of the embodiments.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An ultrasound system, comprising:
an ultrasound data acquisition unit including an ultrasound probe for transmitting ultrasound signals to a target object and receiving ultrasound echo signals reflected from the target object, the ultrasound data acquisition unit being configured to acquire ultrasound data based on the ultrasound echo signals, the ultrasound data acquisition unit being further configured to detect an orientation and a scanning direction of the ultrasound probe; and
a processor for forming a volume data based on the ultrasound data and forming an orientation help (OH) view showing a spatial orientation of volume data on a display region, the OH view including a probe orientation marker indicating the detected orientation of the ultrasound probe and a scanning direction marker indicating the detected scanning direction.

2. The ultrasound system of claim 1, wherein the scanning direction marker includes a first scanning direction marker indicating a swing direction of an array transducer of the ultrasound probe and a second scanning direction marker indicating a scan line direction.

3. The ultrasound system of claim 1, wherein the processor comprises:
a volume data forming section for forming the volume data based on the ultrasound data;
an ultrasound image forming section for forming a three-dimensional ultrasound image and images of reference planes of the three-dimensional ultrasound image; and
an OH view forming section for forming the OH view.

4. The ultrasound system of claim 3, further comprising:
a user interface for receiving a first user instruction for setting a region of interest (ROI) of the three-dimensional ultrasound image, a second user instruction for selecting one of the reference planes and a third user instruction for setting a geometrical operation of the OH view.

5. The ultrasound system of claim 4, wherein the geometrical operation includes rotation, reduction, enlargement and movement of the OH view.

6. A method of providing an orientation help (OH) view in an ultrasound system including an ultrasound probe comprising:
a) obtaining ultrasound data by an ultrasound data acquisition unit within the ultrasound system;
b) detecting an orientation and a scanning direction of the ultrasound probe by the ultrasound data acquisition unit;
c) forming a volume data based on the ultrasound data by a processor within the ultrasound system; and
d) forming an orientation help (OH) view showing a spatial orientation of volume data on a display region, the OH view including a probe orientation marker indicating the detected orientation of the ultrasound probe and a scanning direction marker indicating the detected scanning direction by the processor.

7. The method of claim 6, wherein the scanning direction marker includes a first scanning direction marker indicating a swing direction of an array transducer of the ultrasound probe and a second scanning direction marker indicating a scan line direction.

8. The method of claim 6, further comprising:
e) forming a three-dimensional ultrasound image and images of reference planes of the three-dimensional ultrasound image by the processor;
f) setting a region of interest (ROI) of the three-dimensional ultrasound image through user instruction by a user interface within the ultrasound system;
g) selecting one of the reference planes through user instruction by the user interface; and
h) setting a geometrical operation of the OH view through user instruction by the user interface.

9. The method of claim 8, wherein the geometrical operation includes rotation, reduction, enlargement and movement of the OH view.

10. A computer readable medium comprising instructions that, when executed by a processor, perform a method of providing orientation help (OH) view in an ultrasound system including an ultrasound probe, causing the processor to perform steps comprising:
a) obtaining ultrasound data;
b) detecting an orientation and a scanning direction of a ultrasound probe;
c) forming a volume data based on the ultrasound data; and
d) forming an orientation help (OH) view showing a spatial orientation of volume data on a display region, the OH view including a probe orientation marker indicating the detected orientation of the ultrasound probe and a scanning direction marker indicating the detected scanning direction.

11. The computer readable medium of claim 10, wherein the scanning direction marker includes a first scanning direction marker indicating a swing direction of an array transducer of the ultrasound probe and a second scanning direction marker indicating a scan line direction.

12. The computer readable medium of claim 11, further comprising instructions that, when executed by a processor, cause the processor to perform steps comprising:
e) forming a three-dimensional ultrasound image and images of reference planes of the three-dimensional ultrasound image;
f) setting a region of interest (ROI) of three-dimensional ultrasound image;
g) selecting one of the reference planes; and
h) setting a geometrical operation of the OH view.

13. The computer readable medium of claim 12, wherein the geometrical operation includes rotation, reduction, enlargement and movement of the OH view.
